(19) United States
(12) Patent Application Publication
(10) Pub. No.: US 2005/0101660 A1
(43) Pub. Date: May 12, 2005

(54) Composition for prevention and treatment of obesity, cardiovascular and coronary artery disease

(75) Inventor: Haeng Woo Lee, Seoul (KR)

Correspondence Address: MAYER, BROWN, ROWE & MAW LLP, 190 SOUTH LASALLE ST, CHICAGO, IL, 60603-3441, US

(73) Assignee: THE SKINNY DRINK COMPANY, Tukwila, WA (US)

(21) Appl. No.: 10/769,822

(22) Filed: Feb. 2, 2004

(30) Foreign Application Priority Data
Nov. 11, 2003 (KR) 10-2003-83137

(51) Int. Cl. A61K 31/335

(52) U.S. Cl. 514/452

(57) ABSTRACT

Compositions for the prevention and treatment of obesity, cardiovascular and coronary artery diseases are provided. The present invention provides compositions comprising at least one dibenzo-p-dioxine derivatives having inhibition ability against diacylglycerol acyltransferase (DGAT), and also provides the use of the compositions as a component of drugs or dietary supplements intended for the prevention and treatment of obesity, cardiovascular and coronary artery diseases. Since the compositions of the invention are nontoxic, allowing their chronic ingestion, easier and natural control of obesity and related diseases frequently found in obese people can be realized.

### CROSS-REFERENCE TO RELATED APPLICATIONS

This application claims priority to Korean patent application 10-2003-83137, filed Nov. 11, 2003, incorporated herein by reference in its entirety.

### BACKGROUND OF THE INVENTION

In the modern age, which is characterized by excessive nutrition and lack of exercise due to modern-day conveniences, such as, for example, automobiles, elevators, and convenience foods, the energy balance inside human beings is out of balance, resulting in increased incidence of obesity, diabetes, cardiovascular and coronary artery disease, hyperlipemia, and diabetes, and creating serious social problems. About 20% of the US population currently suffers from obesity, which is generally defined in humans as a body mass index (BMI) higher than 30 kg/m2. Obesity is a major risk factor for hypertension, diabetes and cardiovascular diseases. Annually, about 100 billion dollars are spent to control and fight against obesity. Yet, there is no easy and effective remedy for the treatment of obesity to date, despite the high demand and the availability of many drugs and supplements claiming obesity control.

Obesity can be viewed as an energy storage disorder. Weight gain results from an energy imbalance (i.e.,energy input exceeding output), with most excess calories stored as triglycerides in adipose tissue. A key enzyme in triglyceride synthesis is acyl CoA:diacylglycerol acyltransferase (DGAT), a microsomal enzyme that is widely expressed in mammalian tissues. Cases, et. al., Proc. Natl. Acad. Sci. USA, 95:13018-13023 (October 1998). DGAT catalyzes the final reaction in the glycerol phosphate pathway, which is considered the main pathway of triglyceride synthesis in cells. The enzyme is also believed to catalyze the final step of the monoacylglycerol pathway found predominantly in enterocytes of the small intestine.

Recently, inactivation of the DGAT gene in mice has been studied. Surprisingly, DGAT knockout (Dgat−/−) mice, although deficient in DGAT activity, were viable and capable of synthesizing triglycerides, as evidenced by normal fasting serum triglyceride levels and normal adipose tissue composition. Smith S J, Cases S, Jensen D R, et al. Nat Genet 25:87-90 (2000). When fed a regular chow diet (4% fat by weight), the knockout mice exhibited weight curves similar to those of wild-type mice. Dgat−/− mice, however, had less adipose tissue, as reflected by lower total fat pad weights and body triglyceride content. Furthermore, when the mice were fed a high-fat diet (21% fat by weight), DGAT wild-type and heterozygous mice increased their weight by 40-50%, but Dgat−/− mice maintained weights comparable to those of mice fed the regular chow diet. Subsequent studies on the mechanism by which the suppression of DGAT activity is related to obesity resistance have been performed to reveal that DGAT deficiency protects against obesity in part by enhancing physical activity in response to increased fat intake, thereby “burning off” the additional caloric intake. Chen & Farese, Jr., TCM Vol. 10, No. 5 (2000). Therefore, the suppression of DGAT activity is expected to contribute to obesity control as well as to the prevention of circulatory diseases such as cardiovascular disease, hyperlipemia and diabetes, and other obesity-related conditions.

Thus, there is a high demand for safe agents, such as, e.g., food or supplements, useful for the treatment of obesity and related conditions. As such, it is desirable to have a safe composition for the prevention and treatment of obesity and related diseases, and for the treatment of cardiovascular and coronary artery disease that has the ability to inhibit the DGAT pathway.

### SUMMARY OF THE INVENTION

This invention relates to compositions for the prevention and treatment of obesity and related conditions, and cardiovascular and coronary artery diseases. In one embodiment, the present invention relates to the composition comprising dibenzo-p-dioxine derivatives having inhibition ability against diacylglycerol acyltransferase (DGAT), and also to the use of such compositions as a component of drugs or dietary supplements suitable for the prevention and treatment of obesity, cardiovascular and coronary artery diseases.

### DETAILED DESCRIPTION OF THE INVENTION

The present invention provides compositions useful for the prevention and treatment of obesity, cardiovascular and coronary artery diseases and hyperlipemia. Such compositions are safe for administration to animals and human beings. In one embodiment, compositions effective in obesity treatment and control through suppression of the DGAT activity are provided. In one embodiment, the present invention provides dietary supplements effective for obesity control. In one embodiment, the present invention provides dietary supplements effective for the treatment and prevention of cardiovascular and coronary artery disease.

In one embodiment, the present invention provides compositions that suppress DGAT activity. In one embodiment, the compositions of the invention are dibenzo-p-dioxine derivatives. The inventor has discovered that dibenzo-p-dioxine derivatives suppress DGAT activity and result in decrease of weight and body fat, increase of muscle mass, improvement of cholesterol metabolism, and recovery of vasodilatory functions, etc. Such dibenzo-p-dioxine derivatives are useful in the prevention and treatment of obesity, cardiovascular and coronary artery diseases, and other obesity-related conditions.

The dibenzo-p-dioxine derivatives contained in the compositions of this invention are chemical compounds demonstrating biological activities such as, e.g., antioxidant activity, for example, as described in U.S. Pat. No. 6,384,085 and Kang, et. al. Arch. Pharm. Res., 26:286-293 (2003); antiplasmin inhibition, e.g., as described in Fukuyama, et. al., Chem. Pharm. Bull., 38:133-135 (1990), Nakayama, et. al., Agric. Biol. Chem., 53:3025-3030 (1989), Fukuyama, et. al., Chem. Pharm. Bull., 37:2438-2440 (1989), Fukuyama, et. al., Chem. Pharm. Bull., 37: 349-353 (1989), Glombitza, et al., Phytochemistry, 24:543-551 (1985), Fukuyama, et. al., Japan Patent Nos. 58-118580, 118581, 118591, 146579, 188874 (1983), and antibacterial activity, e.g., as described in Nagayama, et. al., J. Antimicrobial Chemotherapy, 50:889-893 (2002). Such dibenzo-p-dioxine derivatives were originally found in species of large brown seaweeds. In the present invention, the dibenzo-p-dioxine derivatives have been confirmed experimentally to have excellent DGAT suppression activity and administration results in subsequent reduction of the amount of adipose tissue and decrease of weight and body fat, increase of muscle mass, improvement of cholesterol, and recovery of vasodilatory functions.

The dibenzo-p-dioxine derivatives of the present invention can be any suitable dibenzo-p-dioxine. In one embodiment, the dibenzo-p-dioxine derivatives have one of the following chemical formulas: 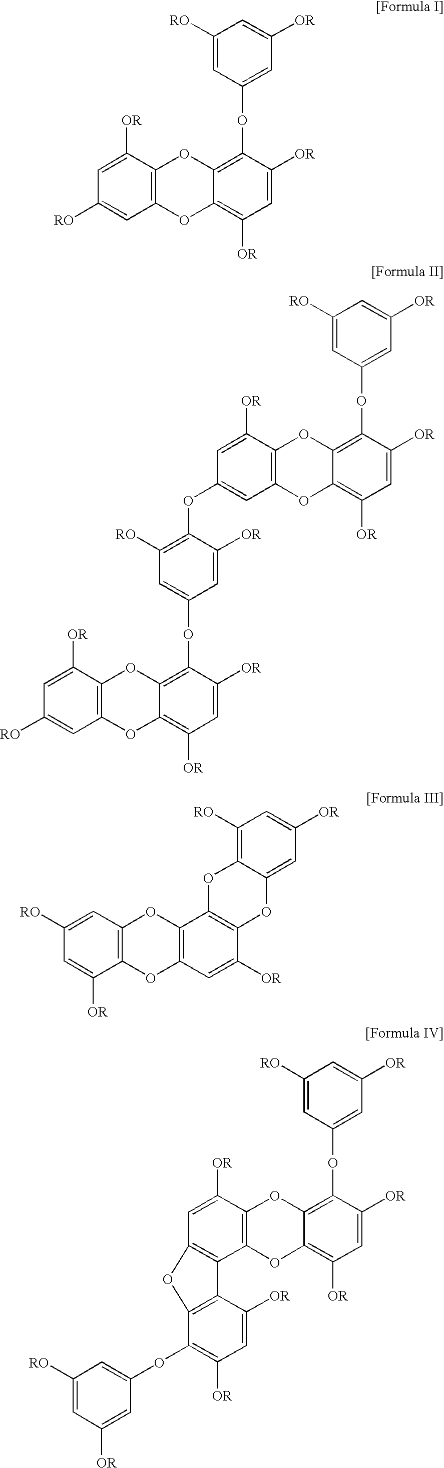 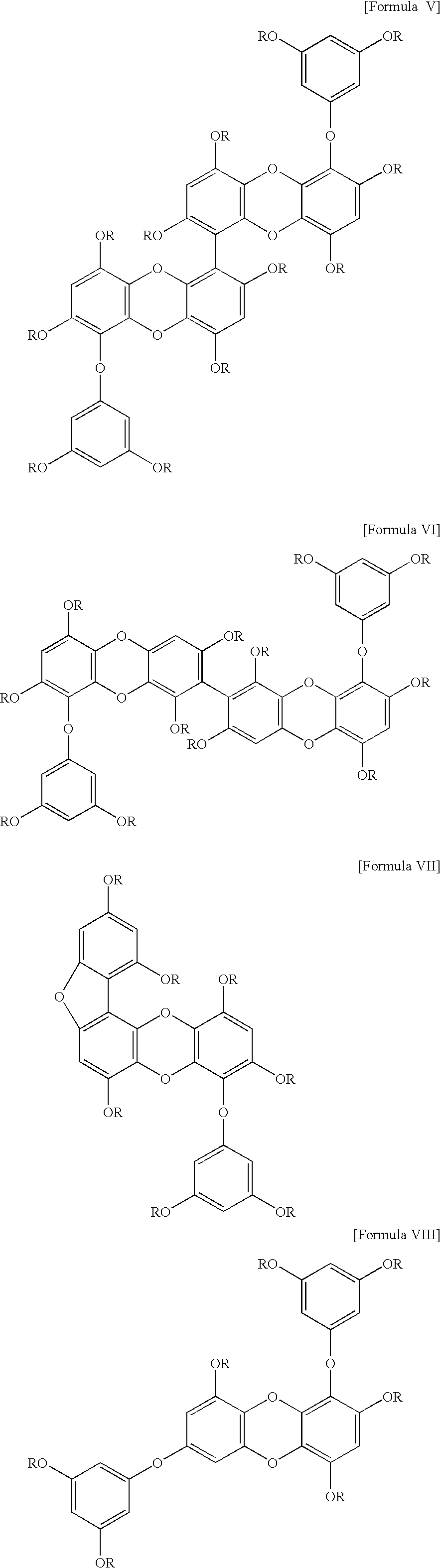 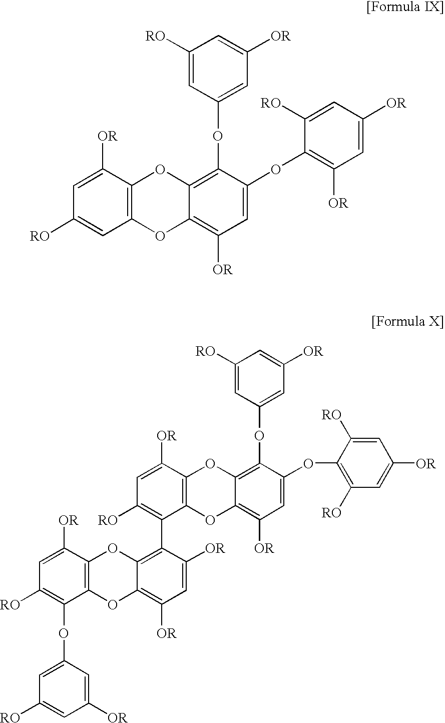
wherein each R is H, alkyl, alkenyl, phenyl, phenylalkyl, alkanoyl, hydroxyphenyl, dihydroxyphenyl or acyl. In one embodiment, each R is H.

The dibenzo-p-dioxin derivatives can be included in the compositions of the invention as a single compound or combinations of two or more of such compounds, such as, e.g., three or more, four or more, five or more, six or more, seven or more, eight or more, nine or more, or ten compounds. For example, in one embodiment, a composition of the invention can comprise two or more of formula I, formula II, formula III, formula IV, formula V, formula VI, formula VII, formula VIII, formula IX, and formula X.

In one embodiment, the composition of the invention comprises one or more of 0.1-6 wt % of the dibenzo-p-dioxin derivative of formula I; 5-60 wt % of the dibenzo-p-dioxin derivative of formula II; 1-30 wt % of the dibenzo-p-dioxin derivative of formula III; 0.5-20 wt % of the dibenzo-p-dioxin derivative of formula IV; 0.1-10 wt % of the dibenzo-p-dioxin derivative of formula V; 0.5-15 wt % of the dibenzo-p-dioxin derivative of formula VI; 0.1-5 wt % of the dibenzo-p-dioxin derivative of formula VII; 0.1-5 wt % of the dibenzo-p-dioxin derivative of formula VIII; 0.1-10 wt % of the dibenzo-p-dioxin derivative of formula IX; or 0.1-12 wt % of the dibenzo-p-dioxin derivative of formula X.

In one embodiment, the composition of the invention comprises two or more of 0.1-6 wt % of the dibenzo-p-dioxin derivative of formula I; 5-60 wt % of the dibenzo-p-dioxin derivative of formula II; 1-30 wt % of the dibenzo-p-dioxin derivative of formula III; 0.5-20 wt % of the dibenzo-p-dioxin derivative of formula IV; 0.1-10 wt % of the dibenzo-p-dioxin derivative of formula V; 0.5-15 wt % of the dibenzo-p-dioxin derivative of formula VI; 0.1-5 wt % of the dibenzo-p-dioxin derivative of formula VII; 0.1-5 wt % of the dibenzo-p-dioxin derivative of formula VIII; 0.1-10 wt % of the dibenzo-p-dioxin derivative of formula IX; or 0.1-12 wt % of the dibenzo-p-dioxin derivative of formula X.

In one embodiment, the composition comprises three or more; four or more; five or more; six or more; seven or more; eight or more; nine or more; or ten of 0.1-6 wt % of the dibenzo-p-dioxin derivative of formula I; 5-60 wt % of the dibenzo-p-dioxin derivative of formula II; 1-30 wt % of the dibenzo-p-dioxin derivative of formula III; 0.5-20 wt % of the diberizo-p-dioxin derivative of formula IV; 0.1-10 wt % of the dibenzo-p-dioxin derivative of formula V; 0.5-15 wt % of the dibenzo-p-dioxin derivative of formula VI; 0.1-5 wt % of the dibenzo-p-dioxin derivative of formula VII; 0.1-5 wt % of the dibenzo-p-dioxin derivative of formula VIII; 0.1-10 wt % of the dibenzo-p-dioxin derivative of formula IX; or O.1-12 wt % of the dibenzo-p-dioxin derivative of formula X.

In one embodiment, the composition comprises: 1-5 wt % of the dibenzo-p-dioxin derivative of formula I; 20-70 wt % of the dibenzo-p-dioxin derivative of formula II; 5-15 wt % of the dibenzo-p-dioxin derivative of formula III; 5-20 wt % of the dibenzo-p-dioxin derivative of formula IV; 5-20 wt % of the dibenzo-p-dioxin derivative of formula V; 5-20 wt % of the dibenzo-p-dioxin derivative of formula VI; 1-5 wt % of the dibenzo-p-dioxin derivative of formula VII; 1-5 wt % of the dibenzo-p-dioxin derivative of formula VIII; 1-10 wt % of the dibenzo-p-dioxin derivative of formula IX; or 1-12 wt % of the dibenzo-p-dioxin derivative of formula X.

In one embodiment, the composition comprises: 1-5 wt % of the dibenzo-p-dioxin derivative of formula I; 50-70 wt % of the dibenzo-p-dioxin derivative of formula II; 5-15 wt % of the dibenzo-p-dioxin derivative of formula III; 10-20 wt % of the dibenzo-p-dioxin derivative of formula IV; 3-10 wt % of the dibenzo-p-dioxin derivative of formula V; 5-15 wt % of the dibenzo-p-dioxin derivative of formula VI; 0-5 wt % of the dibenzo-p-dioxin derivative of formula VII; 0-5 wt % of the dibenzo-p-dioxin derivative of formula VIII; 0-10 wt % of the dibenzo-p-dioxin derivative of formula IX; or 0-12 wt % of the dibenzo-p-dioxin derivative of formula X.

In one embodiment, the composition comprises: 0-5 wt % of the dibenzo-p-dioxin derivative of formula I; 50-70 wt % of the dibenzo-p-dioxin derivative of formula II; 20-40 wt % of the dibenzo-p-dioxin derivative of formula III; 10-20 wt % of the dibenzo-p-dioxin derivative of formula IV; 0-10 wt % of the dibenzo-p-dioxin derivative of formula V; 0-10 wt % of the dibenzo-p-dioxin derivative of formula VI; 0-5 wt % of the dibenzo-p-dioxin derivative of formula VII; 0-5 wt % of the dibenzo-p-dioxin derivative of formula VIII; 0-10 wt % of the dibenzo-p-dioxin derivative of formula IX; or 0-10 wt % of the dibenzo-p-dioxin derivative of formula X.

In a further embodiment, the composition comprises: 0-5 wt % of the dibenzo-p-dioxin derivative of formula I; 10-30 wt % of the dibenzo-p-dioxin derivative of formula II; 0-10 wt % of the dibenzo-p-dioxin derivative of formula III; 50-80 wt % of the dibenzo-p-dioxin derivative of formula IV; 0-10 wt % of the dibenzo-p-dioxin derivative of formula V; 0-10 wt % of the dibenzo-p-dioxin derivative of formula VI; 0-5 wt % of the dibenzo-p-dioxin derivative of formula VII; 0-5 wt % of the dibenzo-p-dioxin derivative of formula VIII.

In one embodiment, the composition comprises 80-100 wt % formula II. In another embodiment, the composition comprises 80-100 wt % formula IV.

In one embodiment, the composition comprises: 0-5 wt % of the dibenzo-p-dioxin derivative of formula I; 30-80 wt % of the dibenzo-p-dioxin derivative of formula II; 0-10 wt % of the dibenzo-p-dioxin derivative of formula III; 0-10 wt % of the dibenzo-p-dioxin derivative of formula IV; 10-40 wt % of the dibenzo-p-dioxin derivative of formula V; 10-40 wt % of the dibenzo-p-dioxin derivative of formula VI; 0-5 wt % of the dibenzo-p-dioxin derivative of formula VII; 0-5 wt % of the dibenzo-p-dioxin derivative of formula VIII.

Suitable dibenzo-p-dioxin derivatives can be extracted from brown alga, such as, for example, from Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana, Ecklonia wrightii. In one embodiment, the dibenzo-p-dioxin derivatives can be extracted from Eisenia bicyclis, Ecklonia cava, Ecklonia kurome or Ecklonia stolonifera. The dibenzo-p-dioxin derivatives can be extracted by standard procedures known in the art.

The composition of the invention can contain any suitable amount of dibenzo-p-dioxin derivatives. In one embodiment, the composition comprises about 0.01 wt % to about 100 wt % of one or more dibenzo-p-dioxin derivatives. In one embodiment, the composition is a food comprising about 0.01 wt % to about 10 wt % of one or more dibenzo-p-dioxin derivatives. In one embodiment, the composition is a dietary supplement comprising about 10 wt % to about 80wt % of one or more dibenzo-p-dioxin derivatives. In one embodiment, the composition is a dietary supplement comprising about 80 wt % to about 99.99 wt % of one or more dibenzo-p-dioxin derivatives.

The composition of the present invention can be prepared in any suitable dosage form. Suitable dosage forms include, but are not limited to, those suitable for oral, rectal, buccal (for example, sublingual), parenteral (for example, intravenous), topical, ocular, pulmonary or subcutaneous routes of administration. Examples of suitable dosage forms include, e.g., a tablet, a powder, a capsule, a suspension, a syrup, a dietary supplement, a beverage, a food (such as, e.g., a bar or a bread) or any other suitable dosage forms. In one embodiment, the composition is a dietary supplement, such as, e.g., a beverage, such as, for example, an alcoholic beverage, a carbonated beverage, a water, tea or coffee; a capsule; a tablet; or a food, such as, for example, a bar, a bread, a snack, cereal, candy, gum, chocolate, soup, a hamburger patty, a meatball, ham, sausage, pepperoni, salad dressing, a sauce, ice cream, a pop, yogurt, cookies, cakes or any kind of food suitable for ingestion.

Thus, the present compounds can be systemically administered, e.g., orally. They can be enclosed in hard or soft shell gelatin capsules, can be compressed into tablets, or can be incorporated directly with the food or drink of the subject. For oral therapeutic administration, the active compound can be combined with one or more excipients and used in the form of ingestible tablets, buccal tablets, troches, capsules, elixirs, suspensions, syrups, wafers, and the like. In one embodiment, the compositions is prepared as a dietary supplement, such as, e.g., a single serving bar, a single serving can or bottle of beverage or a single serving of powdered beverage for mixing with a liquid. In one embodiment, the composition is administered in the form of a pharmaceutical composition and is administered in combination with a pharmaceutically acceptable vehicle such as, e.g., an inert diluent or an assimilable edible carrier. Of course, any material used in preparing any unit dosage form should be substantially non-toxic in the amounts employed. In one embodiment, the active compound may be incorporated into sustained-release preparations and devices.

The compositions of the invention can comprise any additive or combination of additives. The additive can be any suitable additive, including, for example, a flavoring, such as, e.g., a sweetener or other flavoring; a dietary fiber; a coloring; a nutritional supplement, such as, e.g., a vitamin, a mineral, a herb or herbal extracts; or any other functional ingredient, including, for example, a food or nutrient. The composition can combine one or more additives in any suitable combination. In one embodiment, the composition comprises a flavoring, such as, e.g., apple, banana, barley, bean, berry, broccoli, cactus, carrot, cherry, chocolate, citrus, cocoa, cola, corn, fruit, fruit punch, garlic, ginger, grape, grapefruit, jujube, kiwi, lemon, lime, melon, nuts, nut extracts, oat, onion, orange, pear, pineapple, pine tree leaf, raspberry, rice, seaweed, strawberry, tangerine, tomato, vanilla, vegetable, vegetable extracts, vegetable fermentations, walnut, watermelon, wheat or any other suitable flavoring. In one embodiment, the flavoring is a sweetener, such as, e.g., sucralose, fructose, sucrose, stevia, honey, aspartame, crystalline fructose, dextrose, saccharin, acesulfame K or any other suitable sweetener. In one embodiment, the composition comprises a combination of flavorings, including at least one sweetener. In one embodiment, the composition comprises a coloring agent, such as, e.g., crocine, crocetin, carthamas yellow, anthocyanine or any other suitable coloring agent.

In one embodiment, composition comprises at least one dietary fiber, such as, for example, polydextrose, dextrin, undigestible dextrin, galactomannan, alginate, pectin, fucoidan, oligosaccharides, laminarin or any other suitable dietary fiber. In one embodiment, the additive is a nutritional supplement, such as, e.g., a vitamin, a herb or any other suitable nutritional supplement. In one embodiment, the composition comprises at least one functional ingredient, such as, e.g., carnitine, omega-3 oils such as DHA and EPA, stanol and stanol ester, lycopene, rutein, xylitol, Coenzyme Q10, beta-carotene or flavonoids. In one embodiment, the composition comprises at least one vitamin, such as, e.g., Vitamin C, E, B-complex, folic acid). The composition can also comprise at least one amino acid, including, e.g., L-arginine or L-tryptophan, and/or at least one mineral, such as, e.g., selenium, calcium or zinc). In one embodiment, the composition comprises one or more herbal extracts, such as, e.g., ginseng, gingko biloba, saw palmetto, tea, aloe, St John's Wort, Echinacea or any other suitable herbal extract. In one embodiment, the composition comprises a general food or nutrients, such as, e.g., nuts, vegetable oils, fish oils, vinegars, starch, proteins (e.g., meat, bean proteins, whey proteins, gelatins or protein hydrolysates) or fat.

The dibenzo-p-dioxin derivatives of the present invention can be formulated as a single dosage form or as independent multiple dosage forms. The desired dose can conveniently be presented in a single dose or as divided doses administered at appropriate intervals, for example, as two, three, four or more sub-doses per day. The sub-dose itself may be further divided, e.g., into a number of discrete loosely spaced administrations. The dosage forms can be the same or different.

In one embodiment, the composition can be administered daily at the dose of about 0.05 mg/kg to about 400 mg/kg, preferably, about 0.1 mg/kg to about 100 mg/kg, based on the net intake of dibenzo-dioxin compounds. Owing to the absence of toxicity, they may be used in dietary supplements. Any suitable amount of dibenzo-dioxin compounds can be included in the dietary supplement. In one embodiment, the composition comprises about 0.1 to about 100 wt % (preferably about 10 to about 100 wt %, more preferably, about 50 to about 99.9 wt %) of the dibenzo-dioxin compounds can be included in the dietary supplements.

The composition of the invention, such as, e.g., a dietary supplement, is effective in obesity control and the prevention and treatment of cardiovascular and coronary artery diseases. In obesity control, it is especially effective in the reduction of body fat and weight and increase of muscle mass. In the prevention and treatment of cardiovascular and coronary artery disease, the composition is effective for reduction of LDL cholesterol and triglycerides, increase of HDL cholesterol, and recovery of vasodilatory function. Also, administration of the composition results in a decrease in blood sugar levels and an increase in insulin production.

The present invention is further illustrated by the following examples, which should not be construed as limiting in any way. The practice of the present invention will employ, unless otherwise indicated, conventional techniques that are within the skill of the art.

### EXAMPLE I

### DGAT Inhibition

This example demonstrates the inhibition of DGAT activity in rats upon administration of compositions of the invention.

Male SD rats fed a normal diet for 1 week were killed by decapitation and their livers were removed. The livers (200 g) were homogenized by 10 rapid up and down stroke using a motor-driven, Teflon-glass homogenizer in three volumes of cold medium I (0.25 M sucrose, 1 mM EDTA, 10 mM sucrose, 1 mM EDTA, 10 mM Tris-HCl, pH 7.4). The homogenate was centrifuged at 22000 g for 15 min. The resulting supernatant was centrifuged at 81200 g for 1 h. The pellet was suspended in medium I and centrifuged again at 81200 g for 1 h. The final pellet was resuspended in medium I without EDTA. The microsomal fractions of rat livers were prepared and stored at −80° C. until use.

The reaction mixture contained 175 mM Tris-HCl(ph 8.0), 100-200 μg microsomal protein, 14.5 CM BSA, 30 μM [1-14C]palmitoyl-CoA (0.02 μCi), 8 mM MgCl2, 2.5 mM diisopropyl fluorophosphates, 150 μM 1,2-dioleoy-sn-glycerol and test sample dissolved in 50% EtOH (5 μl) in a total vol. of 200 μl.

The assay was initiated by the addition of rat liver microsomal fraction. After 15-min incubation at 23° C., the reaction was stopped by an addition of 1.2 ml of CHCl3-MeOH (1:2) and lipids were extracted using heptane. The lipids were separated by TLC on Silica gel 60 plates using petrol-Et2O—HOAc (80:20:1)-solvent. The distribution of radioactivity on TLC was analyzed with a radioscanner to determine the amount of [14C]triacylglycerol. Inhibition(%) of DGAT was calculated according to Equation 1, where S, S0, S1 is the radioactivity in presence of test sample, in the absence of test sample, and in the absence of enzyme, respectively.
Inhibition(%)=100*(S0−S)/(S0−S1) EQUATION 1:

TABLE 1
Percent Inhibition of DGAT

| | | % DGAT |
|---|---|---|
| Number | Composition | Inhibition |
| 1 | I (R═H), 99% | 28.2 |
| 2 | II (R═H), 98% | 41.7 |
| 3 | III (R═H), 99% | 36.8 |
| 4 | IV (R═H), 99% | 67.8 |
| 5 | V (R═H), 99% | 55.0 |
| 6 | VI (R═H), 99% | 53.9 |
| 7 | VII (R═H), 99% | 54.0 |
| 8 | VIII (R═H), 99% | 46.7 |
| 9 | IX (R═H), 99% | 49.7 |
| 10 | X (R═H), 99% | 64.4 |
| 11 | II (R═H), 60% + III (R═H), 25% + | 50.7 |
| | IV (R═H), 15% | |
| 12 | IV (R═H), 70% + V (R═H) 8% + | 63.8 |
| | VI (22%) | |
| 13 | IV (R═H), 10% + X (R═H), 80% + | 61.6 |
| | VII (R═H), 10% | |
| 14 | I (R═H), 3% + II (R═H), 60% + | 52.8 |
| | III (R═H), 10% + IV (R═H), 12% + | |
| | V (R═H), 5% + VI (R═H), 10% | |
| 15 | II (R═H), 60% + IV (R═H), 20% + | 55.4 |
| | VI (R═H), 15% + VII (R═H), 5% | |
| 16 | IV(R═acetyl, H (3:7)) | 53.2 |
| 17 | II (R═oleoyl, H (1:9) | 33.9 |
| 18 | VI (R═methyl, H (2:8)) | 45.4 |

As demonstrated by Table 1, administration of compositions 1-1 8 comprising one or more dibenzo-p-dioxin derivatives of Formulas I-X resulted in a % DGAT inhibition in all cases, from a 28.2% reduction upon administration of Composition 1 up to a 67.8% reduction upon administration of Composition 4, with the administration of most compositions resulting in about 40% to about 65% DGAT inhibition.

### EXAMPLE 2

### Rat Subacute Toxicity Test

This example demonstrates that there were no toxic effects were found in rats following 4-week repeated oral administration of Composition #14 in Table 1.

Composition #14 and vehicle were repeatedly administered orally with dose of 400, 133, and 44 mg/kg/group for 4 weeks to evaluate toxicity in rats (10 SD rats, male and female each). Following is the summary of results.
(1) No mortality was observed in response to the test article.
(2) General clinical signs following test article administration were not observed.
(3) No change in body weight was observed in both male and female groups.
(4) Test article per se did not induce the difference in the amount of food and water consumption in all groups.
(5) In all groups, there were no abnormal signs in ophthalmoscopic test.
(6) In all groups, urinalysis did not detect any symptoms relevant to test article toxicity.
(7) In all groups, no toxic effect was observed with hematological tests.
(8) In all groups, no toxic effect was observed with serum biochemistry tests.
(9) No specific abnormality was found in autopsy.
(10) No significant difference of organ weights was found between groups.
(11) No toxic effect was found in pathological anatomy examination following test article administration.

As demonstrated by the results, it is apparent that no toxic effects were found in rats following 4-week repeated oral administration of Composition #14 and that the NOAEL (“No Observed Adverse Effect Level”) is 400 mg/kg/day and above.

### EXAMPLE 3

### Slimming Effect Using Beverage Type

This example demonstrates the slimming effect of administration of a composition of the invention to human subjects.

Initially, 150 volunteers elected to participate in the study after having been explained the purpose and protocol of the study. 141 out of 150 finished the study. 9 dropped out of the evaluation due to their absence for the measurement in the week two.

Subjects were told to drink 1 can of product per day at any time of the day for two weeks. Each can of beverage contained 0.022% of Composition 14 (40 mg/180 mL) and flavor.

Height, weight, muscle and body fat of each volunteer were measured one day before commencement of the study. Measurement of Muscle and body fat was performed by impedence method using “Inbody 3.0” (Biospace) 3.0 ″ (Biospace). Starting values are shown in Table 2.

TABLE 2
Values Before Study

| age | | 16.5 ± 0.5 | (16˜17) |
|---|---|---|---|
| sex | Male | 64 | (45.4%) |
| | female | 77 | (54.6%) |

| | | |
|---|---|---|
| Height (cm) | 168.9 ± 7.6 | (150˜188) |
| Weight (kg) | 76.7 ± 16.0 | (52.8˜134.8) |
| BMI (kg/m2) | 26.8 ± 4.7 | (20˜46) |
| Muscle (kg) | 49.0 ± 10.3 | (22.0˜69.6) |
| Body fat (kg) | 24.9 ± 8.9 | (10.8˜61.5) |

Measurements were taken after two weeks. Values before and after were compared using paired t-test and are shown in Table 3.

TABLE 3
Values Before and After Study

| | before | 2 weeks later | difference | % |
|---|---|---|---|---|
| Weight (kg) | 76.74 ± 16.01 | 75.65 ± 15.82 | −1.09* | − |
| Muscle (kg) | 49.01 ± 10.33 | 50.14 ± 10.16 | +1.13** | + |
| Body fat (kg) | 24.88 ± 8.92 | 23.02 ± 8.88 | −1.86** | − |

*P < 0.01,
**P < 0.001

Thus, this example shows that administration of a composition of the invention to human subjects resulted in a slimming effect, such as, e.g., a decrease in body fat, a decrease in overall weight, and/or an increase in muscle mass.

### EXAMPLE 4

### Improvement of cholesterol Metabolism and Recovery of Vasodilatory Function

This example demonstrates the improvement of cholesterol and sugar metabolism and the recovery of vasodilatory function in human subjects after administration of a composition of the invention.

Forty-one hyperlipidemic volunteers entered the study. Thirty-nine of the volunteers completed the study. Hyperlipidemia was defined as a total cholesterol greater that 220 or an LDL level greater than 130.

Subjects were advised to ingest three bars per day at any time of day for six weeks. The bars were ingested freely. Each bar contained 0.2% of Composition #14 (60 mg/30 g).

Blood biochemistry was assessed both before and after the study. Vasodilatory functions of the brachial artery were evaluated using FMD and NMD measured by 10-MHz|linear phased array. Values obtained before and after the study were compared using paired t-test.

Baseline values obtained prior to commencement of the study are shown in Table

TABLE 4
Values Before and After Study

| age | | 55.6 ± 1.2 |
|---|---|---|
| sex | male | n = 17 |
| | Female | n = 22 |

| | |
|---|---|
| height (cm) | 163.4 ± 7.6 |
| weight (kg) | 65.3 ± 5.59 |
| BMI (kg/m2) | 24.4 ± 1.7 |
| Disease history | 11 of the testees had >50% narrowing in coronary artery |

Values obtained before the study and six weeks later are shown in Table 5. Percent change is also shown in Table 5.

TABLE 5
Values Before and After Study

| | | | | % |
|---|---|---|---|---|
| | before | 6 weeks later | difference | change |
| Total cholesterol | 228.3 ± 6.95 | 224.0 ± 6.08 | −4.3 | −1.9% |
| (mg/dL) | | | | |
| LDL cholesterol | 141.1 ± 6.24 | 135.2 ± 5.64* | −5.9 | −4.2% |
| (mg/dL) | | | | |
| HDL cholesterol | 46.5 ± 1.83 | 50.7 ± 2.04** | +4.2 | +9.0% |
| (mg/dL) | | | | |
| Atherogenic | 3.91 ± 0.15 | 3.42 ± 0.14* | −0.49 | −12.5% |
| index¹ | | | | |
| triglycerides | 215.1 ± 23.5 | 195.4 ± 25.3* | −19.7 | −9.2% |
| (mg/dL) | | | | |

¹Atherogenic index = (total cholesterol − HDL cholesterol)/HDL cholesterol
*p < 0.05,
**p < 0.01(compared with initial values)

Recovery of vasodilatory function at baseline and six weeks later is shown in Table 6.

TABLE 6
Recovery of Vasodilatory Function

| | Non CAD(n = 28) | | ³CAD patients(n = 11) | |
|---|---|---|---|---|
| | Week 0 | Week 6 | Week 0 | Week 6 |
| ¹FMD (%) | 6.09 ± 0.57 | 6.12 ± 0.82 | 5.46 ± 1.70 | 7.83 ± 1.95* |
| ²NMD (%) | 11.5 ± 0.98 | 12.2 ± 1.03 | 7.94 ± 1.09 | 11.8 ± 1.72* |

¹FMD: flow mediated dilation
²NMD: nitroglycerin mediated dilation
³CAD: coronary artery disease
*p < 0.05

Based on this example, it is clear that administration of three bars per day comprising a composition of the invention resulted in the improvement of cholesterol and sugar metabolism and the recovery of vasodilatory function in human subjects.

### EXAMPLE 5

### Improvement of Lipid Metabolism with Dietary Supplement

This example demonstrates the improvement of lipid metabolism in human subjects after administration of a composition of the invention.

23 volunteers entered the study with 2 dropouts. Hyperlipidemia was defined as a total cholesterol greater that 220 or an LDL level greater than 130.

Subjects were advised to ingest 6 capsules (3 capsules 2 hr before lunch, 3 capsules 2 hr after dinner) daily for eight weeks. Each capsule contained 40 mg of Composition #11 in table 1.

Blood biochemistry was assessed both before and after the study. Values obtained before the study and eight weeks later are shown in Table 7.

TABLE 6
Improvement of Lipid Metabolism

| N = 21 | 0 week | 8 week | difference | % change |
|---|---|---|---|---|
| Total Cholesterol (mg/dL) | 258.26 ± 28.11 | 233.43 ± 32.08* | −24.83 | −10 |
| LDL Cholesterol (mg/dL) | 171.13 ± 28.02 | 141.78 ± 34.43* | −29.35 | −17 |
| HDL Cholesterol (mg/dL) | 48.52 ± 12.77 | 50.09 ± 13.16 | +1.57 | +3 |
| TG (mg/dL) | 197.74 ± 132.04 | 179.2 ± 112.69* | −18.54 | −9 |
| Artherogenic Index | 4.32 ± 0.45 | 3.66 ± 0.34* | −0.66 | −15.2% |

*p < 0.01 based on data on 0 week.

As exemplified above, it is clear that administration of a composition of the invention resulted in the improvement of lipid metabolism in human subjects.

The invention has been described in an illustrative manner, and it is to be understood the terminology used is intended to be in the nature of description rather than of limitation. All patents and other references cited herein are incorporated herein by reference in their entirety. Obviously, many modifications, equivalents, and variations of the present invention are possible in light of the above teachings. Therefore, it is to be understood that within the scope of the appended claims, the invention may be practiced other than as specifically described.

1. A composition for the treatment or prevention of obesity, wherein the composition comprises at least one dibenzo-p-dioxine derivative with inhibition activity against diacyglycerol acyl transferase.

2. The composition of claim 1, wherein the dibenzo-p-dioxine derivative or derivatives are selected from the group consisting of: 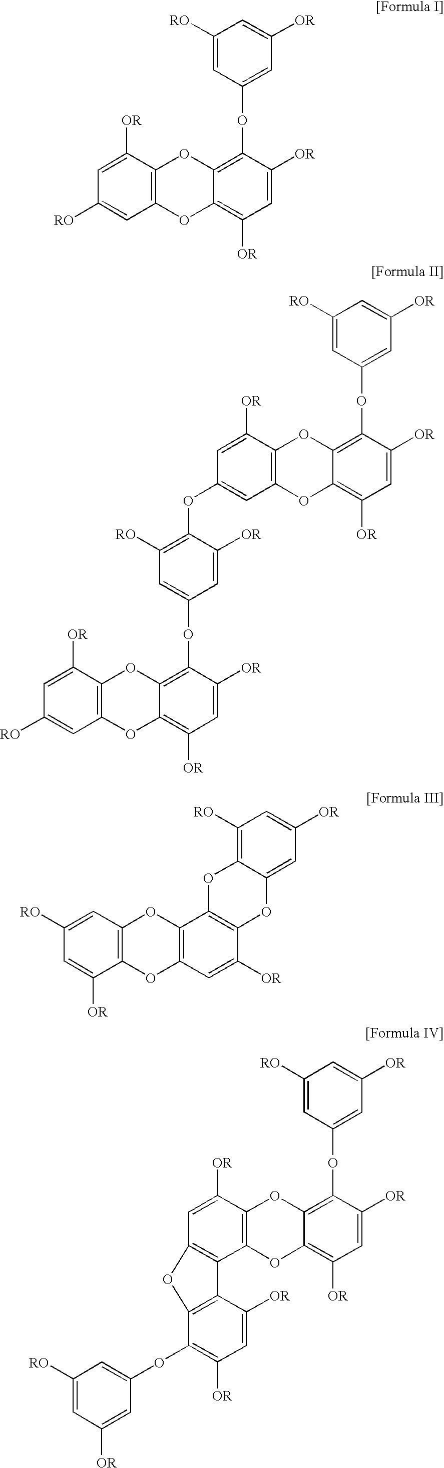 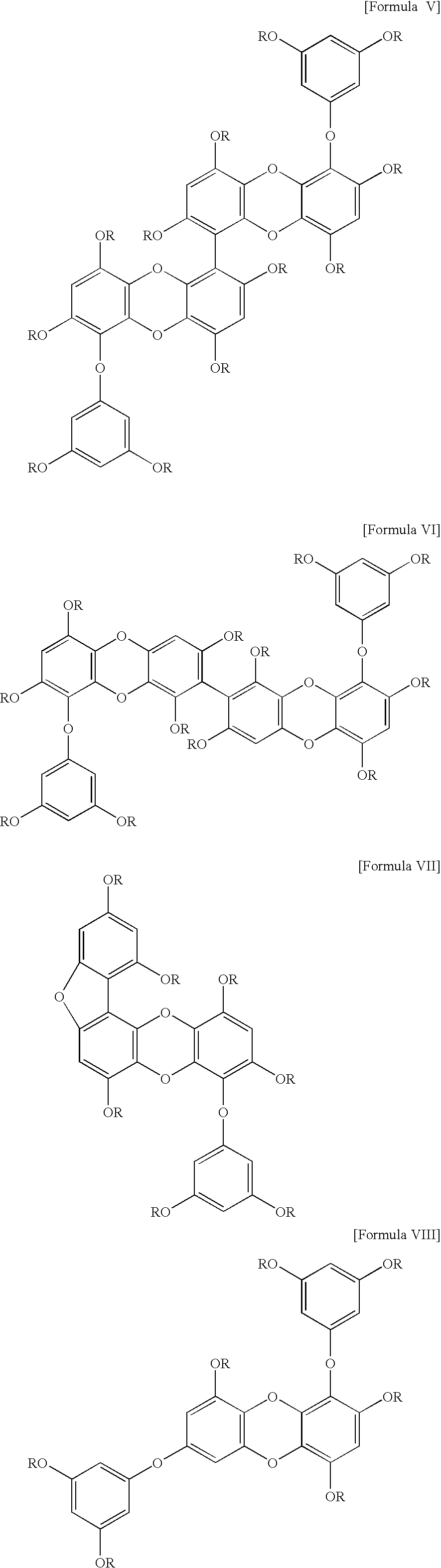 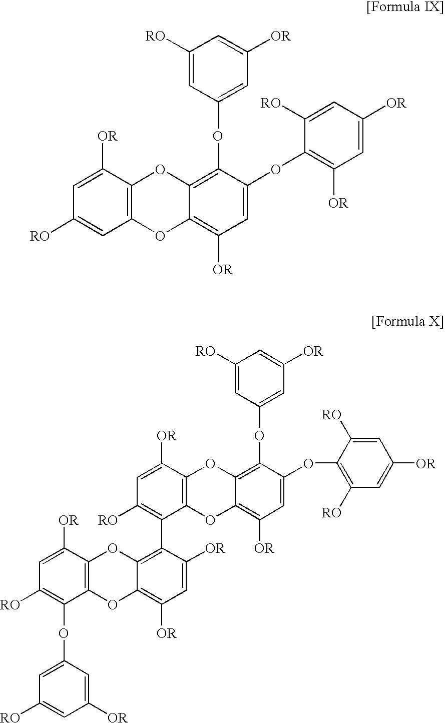
wherein each R is H, alkyl, alkenyl, phenyl, phenylalkyl, alkanoyl, hydroxyphenyl, dihydroxyphenyl or acyl.

3. The composition of claim 2, wherein each R is H.

4. The composition of claim 2, wherein the composition comprises one or more of (a)-(j):
(a) 0.1-6 wt % of the dibenzo-p-dioxin derivative of Formula I;
(b) 5-60 wt % of the dibenzo-p-dioxin derivative of Formula II;
(c) 1-30 wt % of the dibenzo-p-dioxin derivative of Formula III;
(d) 0.5-20 wt % of the dibenzo-p-dioxin derivative of Formula IV;
(e) 0.1-10 wt % of the dibenzo-p-dioxin derivative of Formula V;
(f) 0.5-15 wt % of the dibenzo-p-dioxin derivative of Formula VI;
(g) 0.1-5 wt % of the dibenzo-p-dioxin derivative of Formula VII;
(h) 0. 1-5 wt % of the dibenzo-p-dioxin derivative of Formula VIII;
(i) 0. 1-10 wt % of the dibenzo-p-dioxin derivative of Formula IX; or
(j) 0. 1-12 wt % of the dibenzo-p-dioxin derivative of Formula X.

5. The composition of claim 1, wherein the dibenzo-p-dioxine derivative or derivatives are extracted from brown alga.

6. The composition of claim 5, wherein the brown alga is selected from the group consisting of: Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana, and Ecklonia wrightii.

7. The composition of claim 1, wherein the composition is administered in a daily dosage of 1-100 mg/Kg.

8. The composition of claim 7, wherein the composition is in the form of a dietary supplement.

9. The composition of claim 8, wherein the dietary supplement is in the form of a beverage, a bar, or a bread.

10. The composition of claim 1, wherein the composition is in the form of capsule or tablet.

11. The composition of claim 8, wherein the dietary supplement is in the form of a capsule or tablet.

12. A composition for the treatment or prevention of cardiovascular or coronary artery disease, wherein the composition comprises at least one dibenzo-p-dioxine derivative with inhibition activity against diacyglycerol acyl transferase.

13. The composition of claim 12, wherein the dibenzo-p-dioxine derivative or derivatives are selected from the group consisting of: 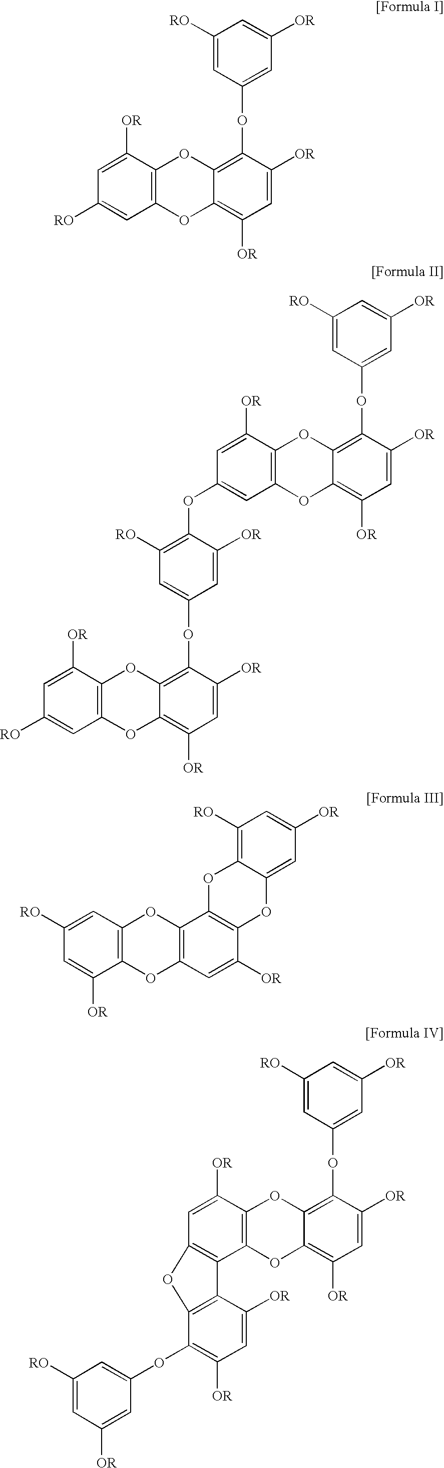 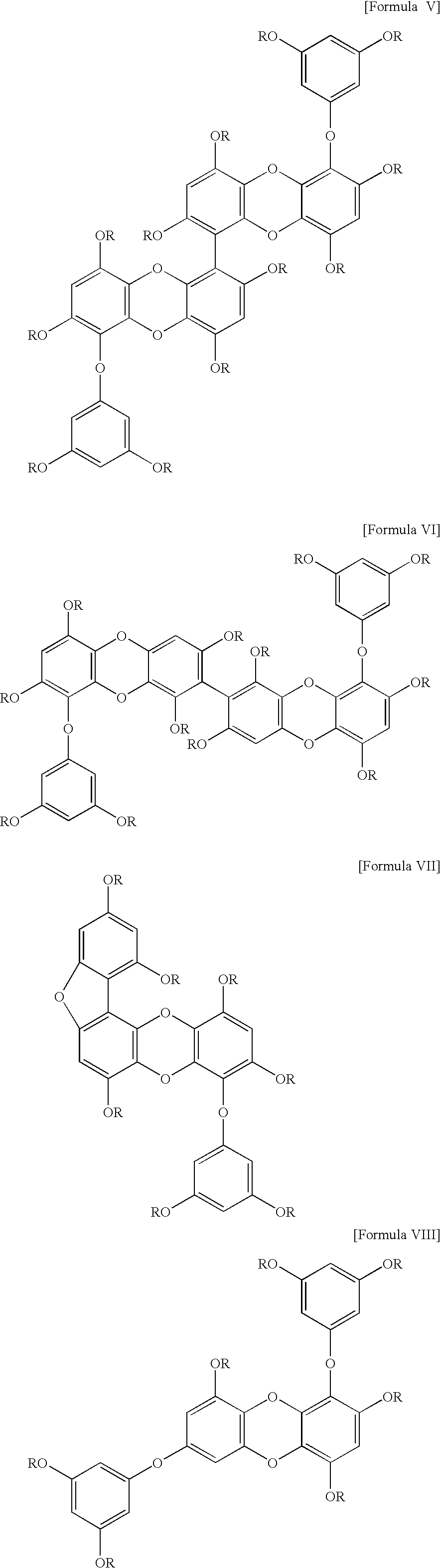 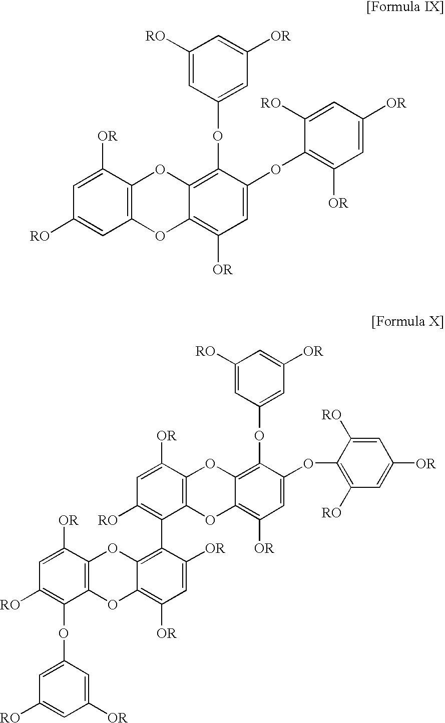
wherein each R is H, alkyl, alkenyl, phenyl, phenylalkyl, alkanoyl, hydroxyphenyl, dihydroxyphenyl or acyl.

14. The composition of claim 13, wherein each R is H.

15. The composition of claim 13, wherein the composition comprises one or more of (a)-(j):
(a) 0.1-6 wt % of the dibenzo-p-dioxin derivative of Formula I;
(b) 5-60 wt % of the dibenzo-p-dioxin derivative of Formula II;
(c) 1-30 wt % of the dibenzo-p-dioxin derivative of Formula III;
(d) 0.5-20 wt % of the dibenzo-p-dioxin derivative of Formula IV;
(e) 0.1-10 wt % of the dibenzo-p-dioxin derivative of Formula V;
(f) 0.5-15 wt % of the dibenzo-p-dioxin derivative of Formula VI;
(g) 0.1-5 wt % of the dibenzo-p-dioxin derivative of Formula VII;
(h) 0.1-5 wt % of the dibenzo-p-dioxin derivative of Formula VIII;
(i) 0.1-10 wt % of the dibenzo-p-dioxin derivative of Formula IX; or
(j) 0.1-12 wt % of the dibenzo-p-dioxin derivative of Formula X.

16. The composition of claim 12, wherein the dibenzo-p-dioxine derivative or derivatives are extracted from brown alga.

17. The composition of claim 16, wherein the brown alga is selected from the group consisting of: Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana, and Ecklonia wrightii.

18. The composition of claim 13, wherein the composition is administered in a daily dosage of 1-100 mg/Kg.

19. The composition of claim 18, wherein the composition is in the form of a dietary supplement.

20. The composition of claim 19, wherein the dietary supplement is in the form of a beverage, a bar, or a bread.

21. The composition of claim 12, wherein the composition is in the form of capsule or tablet.

22. The composition of claim 19, wherein the dietary supplement is in the form of a capsule or tablet.

23. A method of treating or preventing obesity, comprising administering a composition comprising at least one dibenzo-p-dioxine derivative with inhibition activity against diacyglycerol acyl transferase to a subject in need thereof.

24. The method of claim 23, wherein the dibenzo-p-dioxine derivative or derivatives are selected from the group consisting of: 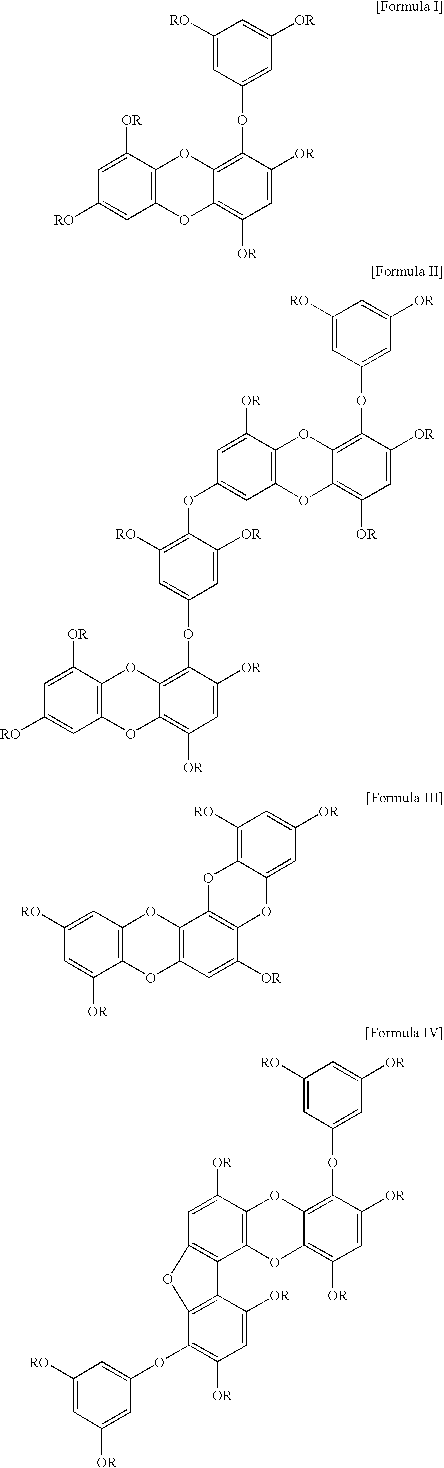 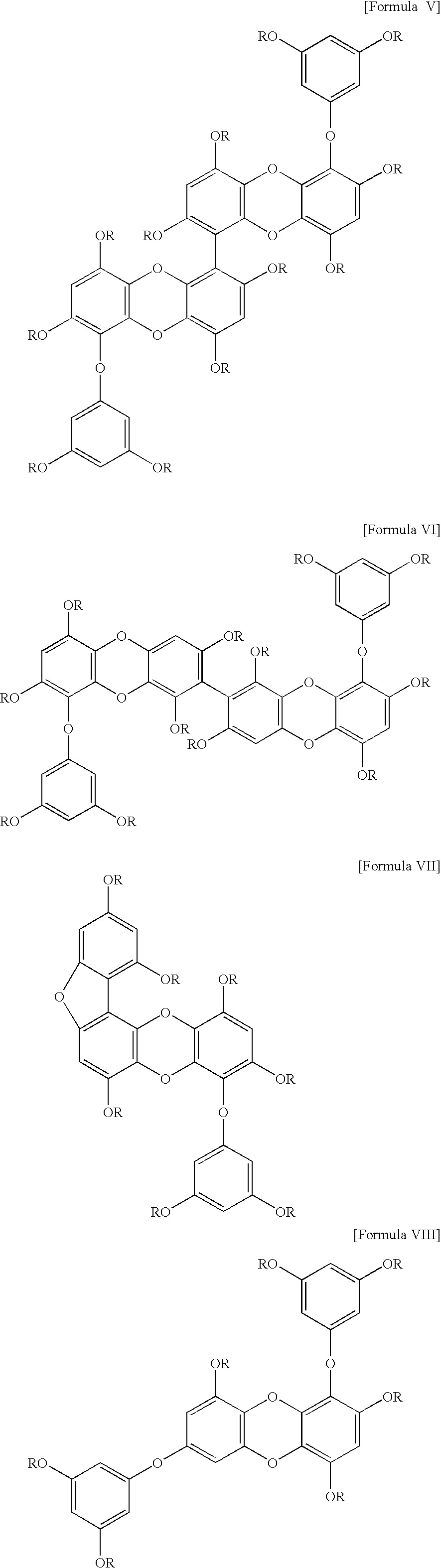 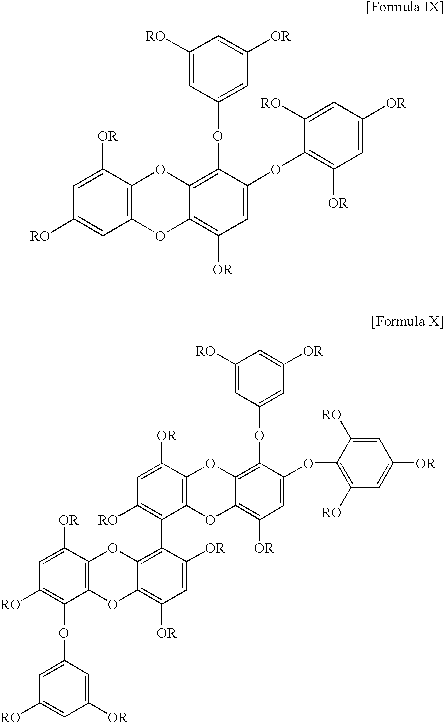
wherein each R is H, alkyl, alkenyl, phenyl, phenylalkyl, alkanoyl, hydroxyphenyl, dihydroxyphenyl or acyl.

25. The method of claim 24, wherein each R is H.

26. The method of claim 24, wherein the composition comprises one or more of (a)-(j):
(a) 0.1-6 wt % of the dibenzo-p-dioxin derivative of Formula I;
(b) 5-60 wt % of the dibenzo-p-dioxin derivative of Formula II;
(c) 1-30 wt % of the dibenzo-p-dioxin derivative of Formula III;
(d) 0.5-20 wt % of the dibenzo-p-dioxin derivative of Formula IV;
(e) 0.1-10 wt % of the dibenzo-p-dioxin derivative of Formula V;
(f) 0.5-15 wt % of the dibenzo-p-dioxin derivative of Formula VI;
(g) 0.1-5 wt % of the dibenzo-p-dioxin derivative of Formula VII;
(h) 0.1-5 wt % of the dibenzo-p-dioxin derivative of Formula VIII;
(i) 0.1-10 wt % of the dibenzo-p-dioxin derivative of Formula IX; or
(j) 0.1-12 wt % of the dibenzo-p-dioxin derivative of Formula X.

27. The method of claim 23, wherein the dibenzo-p-dioxine derivative or derivatives are extracted from brown alga.

28. The method of claim 27, wherein the brown alga is selected from the group consisting of: Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana, and Ecklonia wrightii.

29. The method of claim 23, wherein the composition is administered in a daily dosage of 1-100 mg/Kg.

30. The method of claim 29, wherein the composition is in the form of a dietary supplement.

31. The method of claim 30, wherein the dietary supplement is in the form of a beverage, a bar, or a bread.

32. The method of claim 23, wherein the composition is in the form of capsule or tablet.

33. The method of claim 30, wherein the dietary supplement is in the form of a capsule or tablet.

34. A method for the treatment or prevention of cardiovascular or coronary artery disease, comprising administering a composition comprising at least one dibenzo-p-dioxine derivative with inhibition activity against diacyglycerol acyl transferase to a subject in need thereof.

35. The method of claim 34, wherein the dibenzo-p-dioxine derivative or derivatives are selected from the group consisting of: 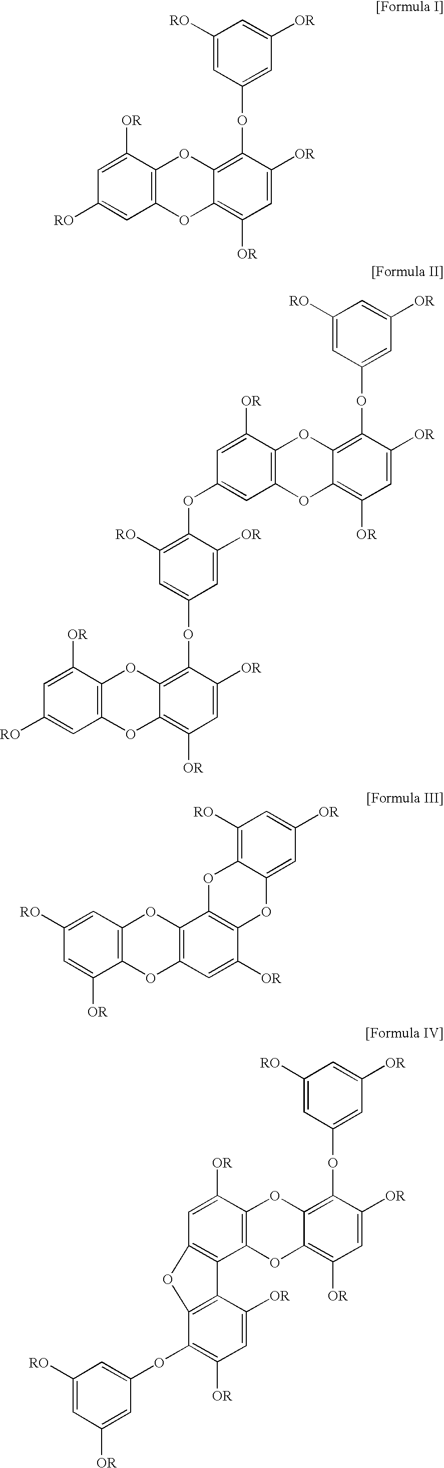 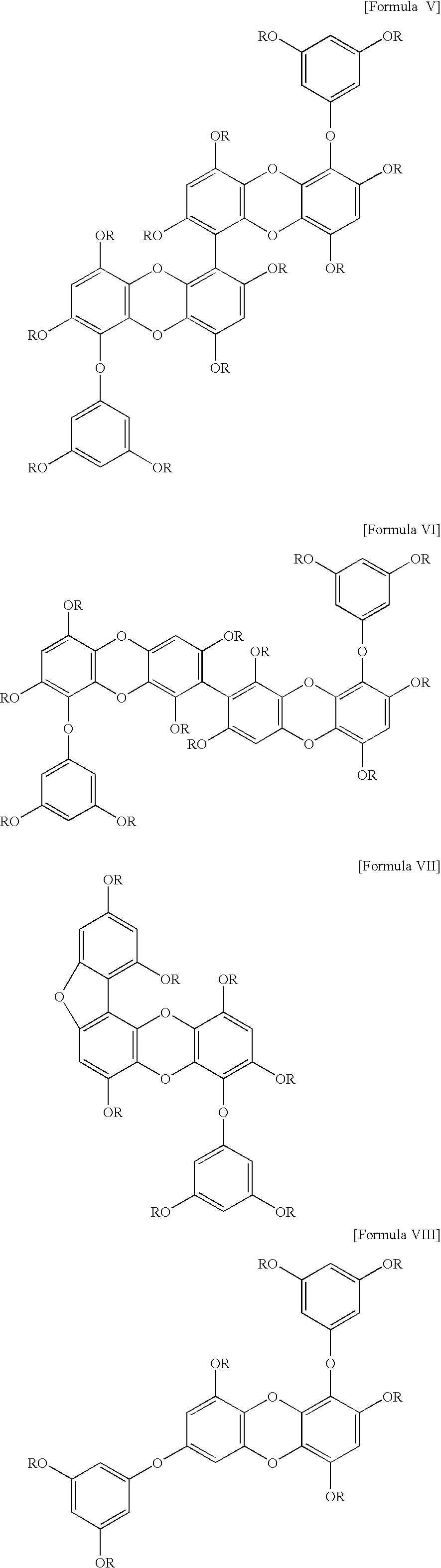 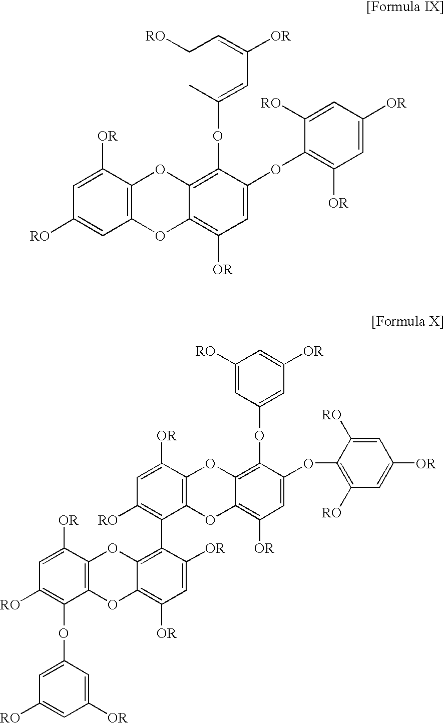
wherein each R is H, alkyl, alkenyl, phenyl, phenylalkyl, alkanoyl, hydroxyphenyl, dihydroxyphenyl or acyl.

36. The method of claim 35, wherein each R is H.

37. The method of claim 35, wherein the composition comprises one or more of (a)-(j):
(a) 0.1-6 wt % of the dibenzo-p-dioxin derivative of Formula I;
(b) 5-60 wt % of the dibenzo-p-dioxin derivative of Formula II;
(c) 1-30 wt % of the dibenzo-p-dioxin derivative of Formula III;
(d) 0.5-20 wt % of the dibenzo-p-dioxin derivative of Formula IV;
(e) 0.1-10 wt % of the dibenzo-p-dioxin derivative of Formnula V;
(f) 0.5-15 wt % of the dibenzo-p-dioxin derivative of Formula VI;
(g) 0.1-5 wt % of the dibenzo-p-dioxin derivative of Formula VII;
(h) 0.1-5 wt % of the dibenzo-p-dioxin derivative of Formula VIII;
(i) 0.1-10 wt % of the dibenzo-p-dioxin derivative of Formula IX; or
(j) 0.1-12 wt % of the dibenzo-p-dioxin derivative of Formula X.

38. The method of claim 34, wherein the dibenzo-p-dioxine derivative or derivatives are extracted from brown alga.

39. The method of claim 38, wherein the brown alga is selected from the group consisting of: Eisenia bicyclis, Eisenia arborea, Eisenia desmarestioides, Eisenia galapagensis, Eisenia masonii, Ecklonia kurome, Ecklonia cava, Ecklonia stolonifera, Ecklonia maxima, Ecklonia radiata, Ecklonia bicyclis, Ecklonia biruncinate, Ecklonia buccinalis, Ecklonia caepaestipes, Ecklonia exasperta, Ecklonia fastigiata, Ecklonia brevipes, Ecklonia arborea, Ecklonia latifolia, Ecklonia muratii, Ecklonia radicosa, Ecklonia richardiana, and Ecklonia wrightii.

40. The method of claim 34, wherein the composition is administered in a daily dosage of 1-100 mg/Kg.

41. The method of claim 40, wherein the composition is in the form of a dietary supplement.

42. The method of claim 41, wherein the dietary supplement is in the form of a beverage, a bar, or a bread.

43. The method of claim 34, wherein the composition is in the form of capsule or tablet.

44. The method of claim 41, wherein the dietary supplement is in the form of a capsule or tablet.
